(19) United States
(12) Patent Application Publication
(10) Pub. No.: US 2014/0127704 A1
(43) Pub. Date: May 8, 2014

(54) KIT FOR DIAGNOSING CARDIOMYOPATHY AND METHOD FOR PREDICTING CARDIOMYOPATHY

(71) Applicants: Show-Li CHEN, Taipei City (TW); Ssu-Yu LIN, Taipei City (TW); Szu-Wei CHANG, Taipei City (TW); Hsin-Hsiung CHEN, Taipei City (TW)

(72) Inventors: Show-Li CHEN, Taipei City (TW); Ssu-Yu LIN, Taipei City (TW); Szu-Wei CHANG, Taipei City (TW); Hsin-Hsiung CHEN, Taipei City (TW)

(73) Assignee: NATIONAL TAIWAN UNIVERSITY, Taipei City (TW)

(21) Appl. No.: 13/669,139

(22) Filed: Nov. 5, 2012

(51) Int. Cl. G01N 33/566; C07K 16/18; C12Q 1/68

(52) U.S. Cl. 435/6.13; 435/7.1; 436/501; 435/6.1; 530/389.8

(57) ABSTRACT

The present invention provides a kit for diagnosing the cardiomyopathy comprising an antibody or oligonucleotide for measuring an expression level of nuclear receptor interaction protein (NRIP). The present invention also provides a method of predicting a risk of cardiomyopathy for a subject. The present invention further provides a method of screening an agent for treating cardiomyopathy.

### FIELD OF THE INVENTION

The present invention relates to a kit for diagnosing cardiomyopathy. This invention also relates to a method for predicting a risk of cardiomyopathy for a subject. This invention further relates to a method for screening an agent for treating cardiomyopathy.

### BACKGROUND OF THE INVENTION

A cardiomyopathy is the deterioration of the function of the myocardium. In cardiomyopathy, the heart muscle becomes enlarged, thick, or rigid. A subject with cardiomyopathy is often at risk of dangerous forms of irregular heart beat, heart failure and sudden cardiac death. According to the classification of pathphysiology, the cardiomyopathy encompasses four forms as follows: hypertrophic cardiomyopathy (HCM), dilated cardiomyopathy, restrictive cardiomyopathy and arrhythmogenic right ventricular cardiomyopathy. The common forms of cardiomyopathy are hypertrophic cardiomyopathy and dilated cardiomyopathy.

HCM is an intrinsic myocardial disorder characterized by unexplained left ventricular hypertrophy. Currently, the prevalence of HCM is about 0.2% to 0.5% of the general population. Many mechanisms and determinants cause the occurring of the disease and sarcomere mutation is most frequently reason among them. In HCM, the sarcomeres in the heart increase in size, which results in the thickening of the heart muscle. In addition, the normal alignment of muscle cells is disrupted, a phenomenon known as myocardial disarray. HCM also causes disruptions of the electrical functions of the heart. HCM is most commonly due to a mutation in one of 9 sarcomeric genes that results in a mutated protein in the sarcomere, the primary component of the myocyte.

A nuclear receptor interaction protein (NRIP) is a transcription factor that only expresses in cell nuclei. It is reported that lack of NRIP gene expression in clinical muscular dystrophy and NRIP knockout mice display weaker muscle strength, indicating that NRIP plays a role in muscle function (Zhang Y et al, Differential expression profiling between the relative normal and dystrophic muscle tissues from the same LGMD patient. J Transl Med, 2006, 4: 53).

The relationship of cardiomyopathy and NRIP is not studied in the previous reports.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1E show that NRIP is a novel α-actinin-2 (ACTN2) binding protein.

FIG. 1A shows direct interaction between NRIP and ACTN2 via GST pull-down assay. The bacterially expressed GST-tagged α-actinin-1 (ACTN1) and ACTN2 proteins are incubated with His-tagged NRIP protein, respectively. Ten percent of the His-NRIP input and the GST-pull-down lysates are separated by SDS-PAGE, and detected by immunoblotting analysis using anti-NRIP antibody (upper). The purified recombinant GST-proteins are stained with Coomassie blue (lower).

FIG. 1B shows that direct interaction between NRIP and ACTN2 is investigated reciprocally via His-tagged pull-down assay.

FIG. 1C shows NRIP deletion mapping for the ACTN2 interaction by pull-down assays. The upper panel represents the schematic map of NRIP protein structure. GST-tagged ACTN2 proteins are incubated with His-tagged NRIP and NRIPΔIQ respectively. The results show IQ domain is responsible for ACTN2 binding.

FIG. 1D shows that ACTN2 and ACTN2 having truncated EF-hand (ACTN2 amino acids 1-750) proteins are incubated with His-tagged NRIP, respectively. The results show that the EF-hand is responsible for NRIP interaction.

FIG. 1E shows that co-immunoprecipitation of NRIP and ACTN2 is performed by using lysates of hearts from wild type (WT, NRIP^{+/+}) and NRIP-null (KO, NRIP^{-/-}) mice.

FIGS. 2A-2F show that NRIP and ACTN2 are colocalized in myocardium.

FIGS. 2A and 2B show that adult (8-10 week old) NRIP^{-/-} mouse myocardium are triple-stained for NRIP (green), α-actinin (red), and nuclei (blue).

FIG. 2C shows that counterstaining with antibody against ACTN2 demonstrates co-localization of NRIP with ACTN2 at cardiac Z-discs.

FIGS. 2D and 2E show that adult (8-10 week old) NRIP^{-/-} mouse myocardium are triple-stained for NRIP (green), myomesin (red), and nuclei (blue).

FIG. 2F shows that counterstaining with myomesin antibody, visualizing A-bands make up of thick myofilaments. Merging of NRIP and myomesin stains reveals localization of NRIP between adjacent A-bands.

FIGS. 3A-3D show hypertrophic response of the heart in NRIP-deficient mice.

FIG. 3A shows that hearts from NRIP^{+/+} and NRIP^{-/-} mice at age 39 weeks were excised following perfused with 0.5% lidocaine and fixed with 4% paraformaldehyde (PFA) at 4° C. overnight. The hearts of NRIP^{-/-} mice are enlarged (upper). Scale bar, 5 mm. Hearts from WT (NRIP^{+/+}) and NRIP-null (NRIP^{-/-}) mice are excised and stained with hematoxylin and eosin (H&E). The left ventricle posterior wall and interventricular septum are thick and the right ventricle is dilated in NRIP^{-/-} mice. Scale bar, 2.0 mm (lower).

FIG. 3B shows that the cell size of NRIP^{-/-} cardiomyocytes are enlarged. The dimension of cell is analyzed by Image J software. Hematoxylin stains nuclei (blue) and eosin stains cytoplasm (red). Scale bar, 20 μm.

FIG. 3C shows the quantitative results of cell size from WT (NRIP^{+/+}) mice and KO (NRIP^{-/-}) mice. NRIP^{+/+} mice (n=2, 182 cells), NRIP^{-/-} mice (n=2, 200 cells).

FIG. 3D shows that the collagen depositions are analyzed with Massion's trichrome staining. Connective tissue is stained blue. Massive collagen deposits in the left ventricle of NRIP^{-/-} mice, which is indicated by arrows. Scale bars, 2.0 mm (Left); 200 μm (Right).

FIGS. 4A-4C show that the cardiac muscle of NRIP-null mice displays abnormal ultrastructure.

The hearts are excised and fixed with 4% paraformaldehyde (PFA) at 4° C. overnight. Some parts of left ventricle posterior wall are used to ultrastructural analysis.

FIG. 4A shows that experiments are performed with myocardium from WT (NRIP^{+/+}) and NRIP^{-/-} mice. Both low (upper, 5000×) and high (lower, 30000×) magnification micrograph shows Z-disc widening and I-band shortening in NRIP deficient mice compared with WT. Scale bars, 2 μm (upper); 500 nm (lower).

FIG. 4B shows magnified images of single sarcomere.

FIG. 4C shows statistical analysis of I-band and Z-disc. The dimensions are measured by Image J software. Data are mean±SE from 10-week old mice. **P<0.01.

### SUMMARY OF THE INVENTION

The present invention provides a kit for diagnosing cardiomyopathy, comprising using an antibody or oligonucleotide to measure the expression level of nuclear receptor interaction protein (NRIP).

The present invention also provides a method of predicting a risk of cardiomyopathy for a subject, comprising measuring the expression level of NRIP in a test sample from said subject, wherein a reduction in the expression level of NRIP in the test sample, relative to the expression level of NRIP in a control sample from a non-cardiomyopathy subject, is indicative of a higher risk of cardiomyopathy for said subject.

The present invention further provides a method of screening an agent for treating cardiomyopathy, comprising providing a test agent to a test sample from subject suffering from cardiomyopathy and measuring the expression level of NRIP, wherein an increasing or equal amount in the level of NRIP in the test sample relative to a control sample from a non-cardiomyopathy subject, is indicative of the test agent being the agent for treating cardiomyopathy.

### DETAILED DESCRIPTION OF THE INVENTION

The present invention shows that nuclear receptor interaction protein (NRIP) is a Z-disc protein of sarcomere. The present invention demonstrates that NRIP interacts with α-actinin-2 (ACTN2) and co-localizes with ACTN2 on Z-bands of cardiac muscle by in vitro and in vivo of protein binding assay and immunohistochemistry. Because the previous reports indicate that ACTN2 belongs a Z-disc protein (Sorimachi H et al, J Mol Biol., 1997, Aug. 1; 270(5):688-695), it demonstrates that NRIP is one of Z-disc proteins according to above results. Further, the results of the present invention from NRIP knockout mice model show that left ventricle of NRIP^{-/-} mice is dilated as compared to NRIP^{+/+} mice and reveal that deficiency of NRIP progressively leads to cardiac hypertrophy.

The present invention shows that NRIP interacts with ACTN2 and is one of Z-disc protein. Moreover, the deficiency of NRIP causes the abnormal cardiac function and leads to hypertrophic cardiomyopathy which is due to left ventricular hypertrophy. According to above results, the present invention can be served as a diagnostic and a therapeutic target for cardiomyopathy or cardiac disease which is caused by deficiency of NRIP. The application of the present invention as follows: to make a pharmaceutical composition of NRIP used to treat cardiomyopathy, to use other agent repairing the abnormal function of NRIP to cure the cardiac disease which was caused by loss of NRIP, and to develop NRIP as a biomarker to identify the occurring risk of cardiomyopathy or progress of cardiac hypertrophy.

As defined herein, the term “cardiomyopathy” refers to a cardiac disease which is induced by loss of NRIP, includes but is not limited to hypertrophic cardiomyopathy, dilated cardiomyopathy, restrictive cardiomyopathy or arrhythmogenic right ventricular cardiomyopathy. The expression level of NRIP, as used herein, is the expression level of protein, RNA or DNA of NRIP. The NRIP, as used herein, is a Z-disc protein and interacts with ACTN2.

The present invention provides a kit for diagnosing cardiomyopathy, comprising an antibody or oligonucleotide for measuring an expression level of NRIP. In a preferred embodiment, the cardiomyopathy is hypertrophic cardiomyopathy.

The present invention also provides a method of predicting a risk of cardiomyopathy for a subject, comprising measuring an expression level of NRIP in a test sample from said subject, wherein a reduction in the expression level of NRIP in the test sample, relative to the expression level of NRIP in a control sample from a non-cardiomyopathy subject, is indicative of a higher risk of cardiomyopathy for said subject. In a preferred embodiment, the said subject and non-cardiomyopathy subject are animal or human. In a preferred embodiment, the test sample and the control sample are blood, serum or plasma.

The present invention further provides a method of screening an agent for treating cardiomyopathy, comprising providing a test agent to a test sample from a subject suffering from cardiomyopathy, and measuring an expression level of NRIP in test sample, wherein an increasing or equal amount in the expression level of NRIP in the test sample, relative to a control sample from a non-cardiomyopathy subject, is indicative of the test agent being the agent for treating cardiomyopathy. In a preferred embodiment, the said subject and non-cardiomyopathy subject are animal or human. In a preferred embodiment, the test sample and the control sample are blood, serum, plasma or cardiac cell.

### EXAMPLES

The examples below are non-limiting and are merely representative of various aspects and features of the present invention.

### Example 1

### The Interacting Partners of NRIP

The present invention used full-length NRIP as bait to perform a yeast two-hybrid screen of a human prostate cDNA library. Under 2×10⁵ of transformants, yeast cells were co-transformed with the bait vector fused to NRIP cDNA and prey vectors fused to the cDNAs from prostate tissues of 25 years old human (Invitrogen). The α-actinin (ACTN) family including ACTN1, ACTN2 and ACTN4 were mostly found from total 145 colonies (Table 1). The result demonstrated that actinins were potentially interacting partners of NRIP.

TABLE 1
Yeast Two-hybrid Library Screening

| | Entrez | | Number |
|---|---|---|---|
| Gene Name | Gene ID | Description | of Colonies |
| ACTN1 | 87 | Non-muscle, cytoskeletal | 39 |
| | | actinin, alpha-1 | |
| ACTN2 | 88 | Muscle, specific actinin, | 10 |
| | | alpha-2 | |
| ACTN4 | 81 | Non-muscle, cytoskeletal | 7 |
| | | actinin, alpha-4 | |
| NR2F2 | 7026 | Nuclear recptor subfamily 2, | 10 |
| | | group F, member 2 | |
| SERTAD1 | 29950 | SERTA domain containing 1 | 4 |
| DNAJA3 | 9093 | DnaJ (Hsp40) homolog, | 1 |
| | | subfamily A, member 3 | |
| eIF4A2 | 1974 | Eukaryotic translation | 1 |
| | | initiation factor 4A2 | |
| H3F3B | 3021 | H3 histone, family 3B | 1 |

### Example 2

### NRIP Interacts with ACTN2 In Vitro and In Vivo

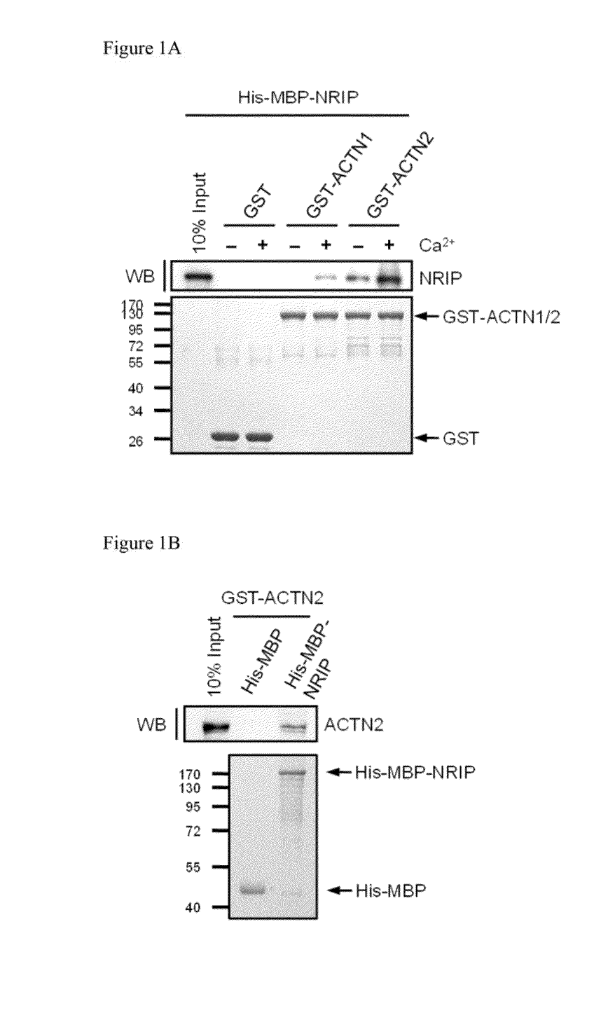

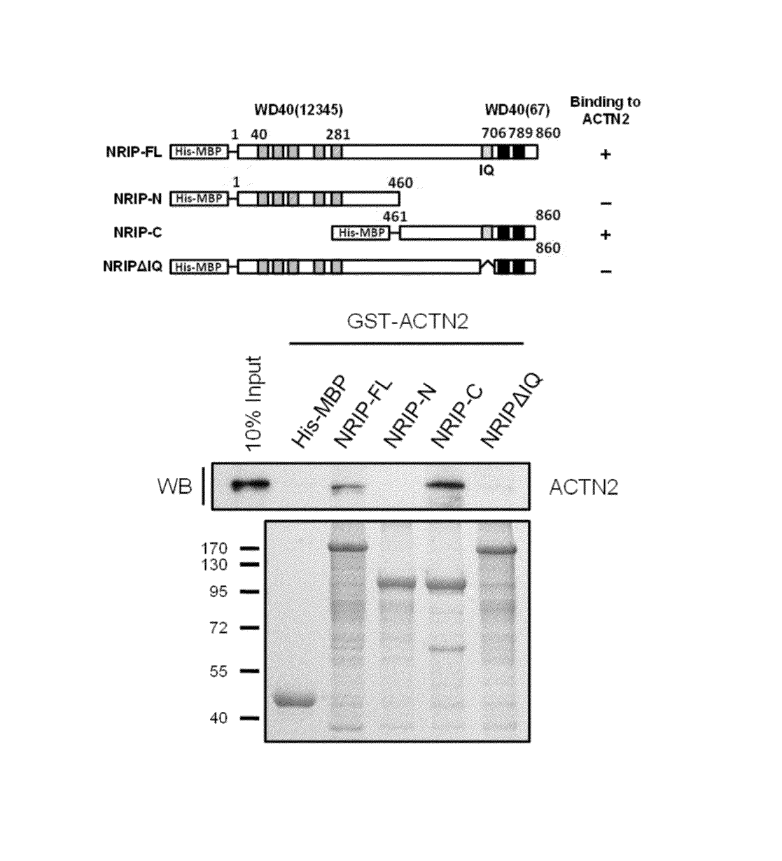

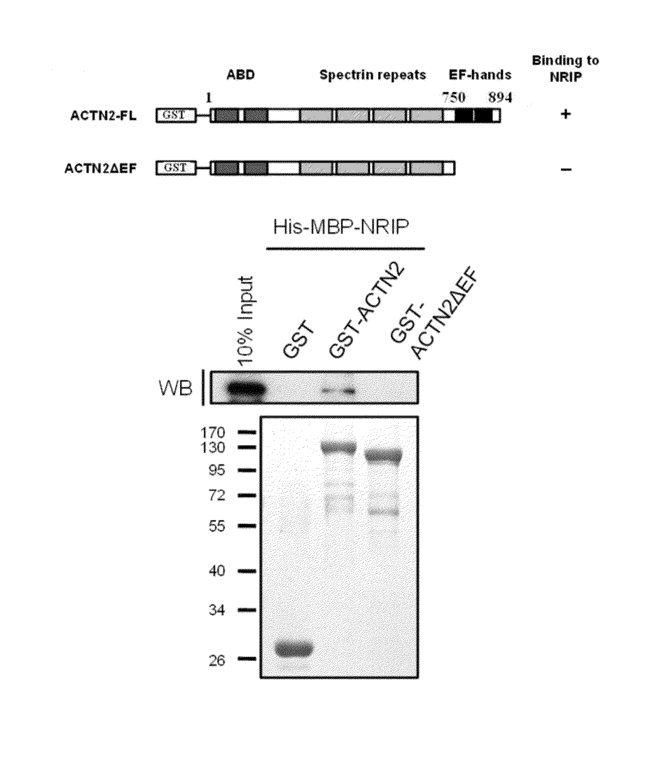

To further confirm the result of yeast two-hybrid assay, the in vitro binding assay using bacterial expressed NRIP and ACTN was performed (FIG. 1A). Both ACTN1 and ACTN2 could interact with NRIP and binding activities were enhanced by adding calcium ion (Ca²⁺) and the result was also confirmed reciprocally (FIG. 1B). Based on previous study, NRIP bound to calmodulin (CaM) in a Ca²⁺-dependent manner through its IQ domain and ACTN2 has a CaM-like EF-hand locating at amino acids 750 to 894 on C-terminus. Therefore, the present invention proposed that the IQ domain of NRIP was able to interact with the EF-hand of ACTN2. Deletion mutants of NRIP and ACTN2 were generated to perform in vitro binding assay. The results showed NRIP interacted with the EF-hand of ACTN2 through the IQ domain (FIGS. 1C and 1D). Moreover, the present invention next examined whether NRIP binds to ACTN2 in vivo by using ACTN2 antibodies to immunoprecipitate lysates of cardiac tissues. The NRIP was able to co-immunoprecipitate with ACTN2 (FIG. 1E), indicating NRIP involves in ACTN2 complex.

### Example 3

### NRIP is a Z-Disc Protein

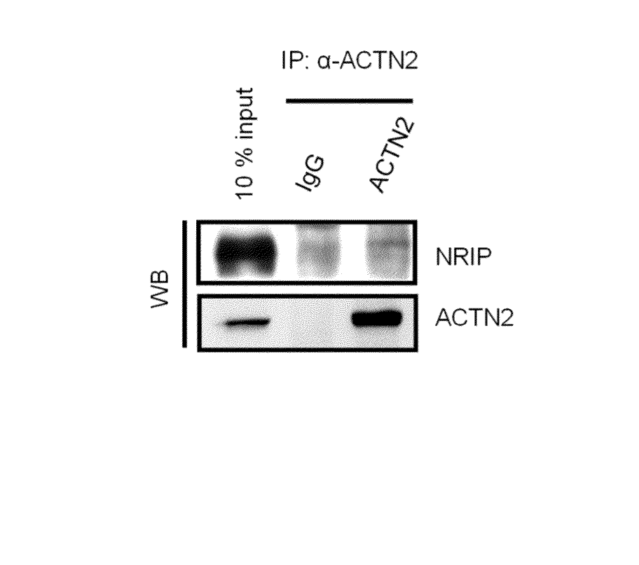

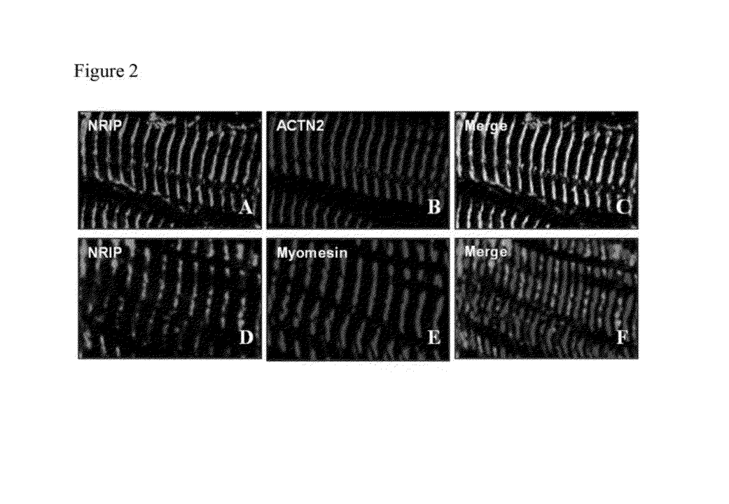

Since α-Actinin was the major component of the Z-disc and ACTN2 was the cardiac muscle-specific isoform, the localization of NRIP at Z-bands in cardiac muscle was further investigated. By using antibodies against NRIP, ACTN2 and myomesin to perform immunofluorescence staining, NRIP was found to co-localize with ACTN2 on Z-bands of cardiac muscle (FIG. 2). The above examples concluded that NRIP is one of Z-disc proteins.

### Example 4

### Loss of NRIP Leads to Defective Cardiac Function

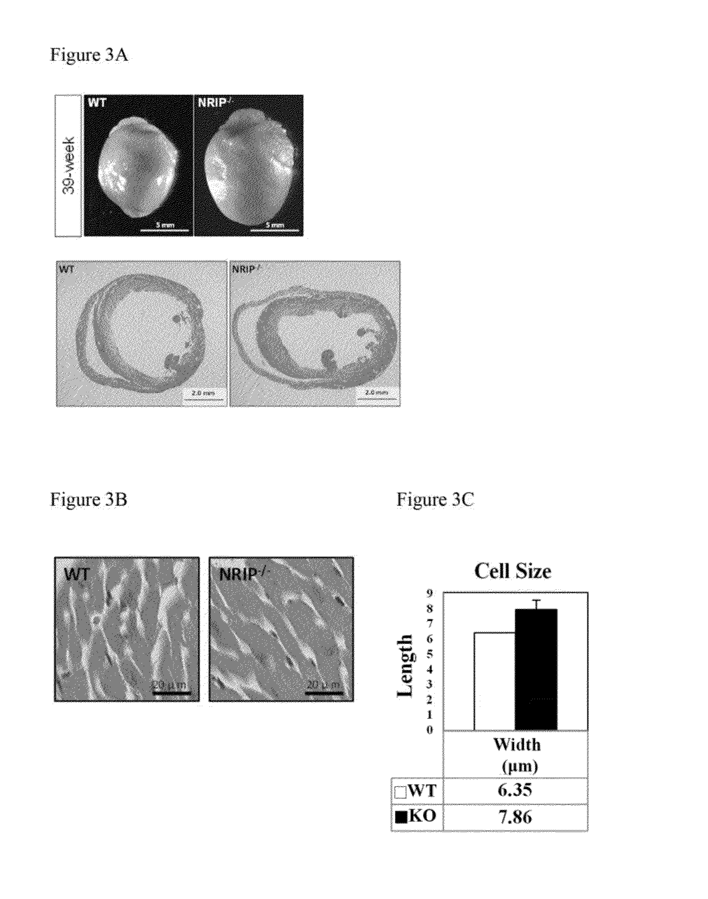

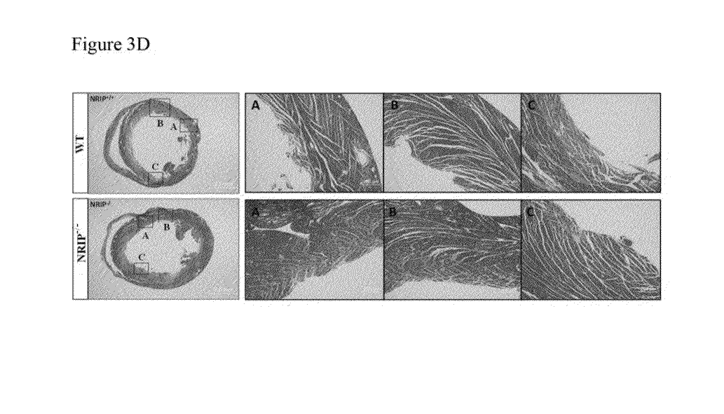

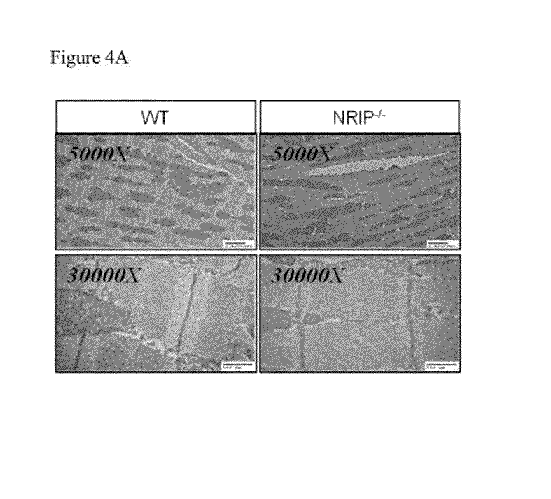

The present invention further examined the cardiac function of wild type (WT, NRIP^{+/+}) and NRIP^{-/-} mice by echocardiography from young to middle aged stage (Table 2). The results showed the measurements of ejection fraction (EF) and fraction shortening (FS) from NRIP^{-/-} mice were lower than WT implying that NRIP^{-/-} mice had cardiac functional defects. Additionally, the dimension of interventricular septum (IVSd), posterior wall (PW) and left ventricle and left ventricle mass were increased with aging showing that NRIP^{-/-} mice were on the process of cardiac hypertrophy. By comparing the hearts excised from WT and NRIP^{-/-} mice at 39 weeks, the sizes of ventricle and atrium of NRIP^{-/-} mice were enlarged (FIG. 3A, upper), and the histological analysis using hematoxylin and eosin (H&E) staining indicates that left ventricle was dilated (FIG. 3A, lower), which were consistent with conclusions of echocardiography. Also, the sizes of cardiomyocytes in 39-week NRIP^{-/-} mice were found to be increased comparing with that in WT (FIG. 3B). Accompanied with cardiac hypertrophy, histological analysis with Massion's trichrome staining revealed significantly more fibrosis in the hearts of NRIP^{-/-} mice compared to WT mice at 39 weeks (FIG. 3C).

TABLE 2
Echocardiographic Analysis of NRIP^{+/+} and NRIP^{-/-} Mice

| | 8-20 weeks old | | 20-30 weeks old | | 30-40 weeks old | |
|---|---|---|---|---|---|---|
| parameters | NRIP^{+/+} | NRIP^{-/-} | NRIP^{+/+} | NRIP^{-/-} | NRIP^{+/+} | NRIP^{-/-} |
| FS | 39.89 ± 4.09 | 32.41 ± 18.34 | 33.06 ± 1.78 | 22.96 ± 10.37 | 33.53 ± 5.09 | 27.37 ± 6.07 |
| (%) | | | | | | |
| EF | 71.15 ± 4.73 | 58.47 ± 26.19 | 62.42 ± 2.81 | 45.35 ± 17.36 | 63.04 ± 6.85 | 53.3 ± 9.66 |
| (%) | | | | | | |
| LVEDD | 3.73 ± 0.28 | 4.52 ± 0.12 | 3.67 ± 0.58 | 4.64 ± 0.1 | 3.52 ± 0.24 | 4.26 ± 0.24* |
| (mm) | | | | | | |
| LVESD | 2.24 ± 0.19 | 3.06 ± 0.92 | 2.51 ± 0.43 | 3.57 ± 0.4* | 2.34 ± 0.28 | 3.09 ± 0.43 |
| (mm) | | | | | | |
| IVSd | 0.97 ± 0.34 | 1.15 ± 0.27 | 0.59 ± 0.11 | 0.85 ± 0.049* | 0.57 ± 0.04 | 0.78 ± 0.09* |
| (mm) | | | | | | |
| IVSs | 0.69 ± 0.24 | 0.69 ± 0.11 | 0.43 ± 0.04 | 0.66 ± 0.028* | 0.47 ± 0.05 | 0.66 ± 0.12* |
| (mm) | | | | | | |
| LVPWd | 1.04 ± 0.17 | 1.015 ± 0.31 | 0.64 ± 0.04 | 0.78 ± 0.049* | 0.63 ± 0.04 | 0.97 ± 0.02** |
| (mm) | | | | | | |
| LVPWs | 0.72 ± 0.12 | 0.63 ± 0.03 | 0.49 ± 0.08 | 0.645 ± 0.049 | 0.51 ± 0.06 | 0.745 ± 0.049* |
| (mm) | | | | | | |
| HR | 381.5 ± 77.25 | 284.5 ± 0.26 | 471 ± 47.14 | 365 ± 65.05 | 511 ± 37.64 | 399 ± 8.48* |
| LVM | 146.7 ± 44.88 | 74.04 ± 0.19 | 73.25 ± 20.91 | 155.36 ± 6.32** | 65.79 ± 13.78 | 147.65 ± 28.11** |
| (mg) | | | | | | |

LV: left ventricle;
FS: fractional shortening of LV;
EF: ejection fraction of LV;
LVEDD: end-diastolic dimension of LV;
LVESD: end-systolic dimension of LV;
IVSD: interventricular septum dimension;
IVSs: interatrial septum dimension;
LVPWd: end-diastolic posterior wall thickness of LV;
LVPWs: end- systolic posterior wall thickness of LV;
FIR: heart rate;
LVM: mass of LV.
*P < 0.05;
**P < 0.01 represent significant differences between the measurements in NRIP^{-/-} mice compared with NRIP^{+/+} at the same age.

### Example 5

### NRIP Deficiency Leads to Abnormal Ultrastructure of Cardiac Muscle

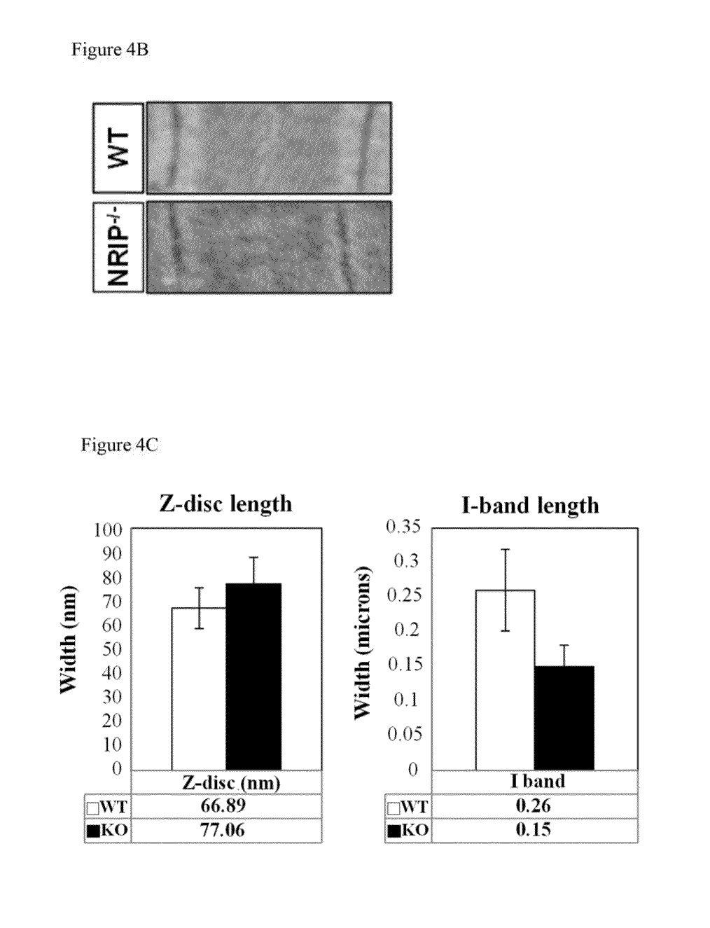

Many Z-disc proteins have proved that mutations or defects of these proteins disrupt cardiac cytoarchitectural organization and lead to cardiomyopathy. The present invention next investigated the effects of NRIP^{-/-} in sarcommeric structure. Hearts were excised from adult WT (NRIP^{+/+}) and KO (NRIP^{-/-}) mice, and fixed for transmission electron microscopy (TEM) analysis (FIG. 4A). The results showed that the width of I-band from NRIP^{-/-} mice was reduced and the Z-disc was widened (FIGS. 4B and 4C).

One skilled in the art readily appreciates that the present invention is well adapted to carry out the objects and obtain the ends and advantages mentioned, as well as those inherent therein. The animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A kit for diagnosing the cardiomyopathy comprising an antibody for measuring an expression level of nuclear receptor interaction protein (NRIP).

2. The kit of claim 1, wherein the cardiomyopathy is hypertrophic cardiomyopathy, dilated cardiomyopathy, restrictive cardiomyopathy or arrhythmogenic right ventricular cardiomyopathy.

3. The kit of claim 2, wherein the cardiomyopathy is hypertrophic cardiomyopathy.

4. The kit of claim 1, wherein the expression level of NRIP is the expression level of protein, RNA or DNA of NRIP.

5. The kit of claim 1, wherein the NRIP is a Z-disc protein.

6. The kit of claim 1, wherein the NRIP interacts with α-actinin-2 (ACTN2).

7. A method of predicting a risk of cardiomyopathy for a subject, comprising measuring an expression level of NRIP in a test sample from said subject, wherein a reduction in the expression level of NRIP in the test sample, relative to the expression level of NRIP in a control sample from a non-cardiomyopathy subject, is indicative of a higher risk of cardiomyopathy for said subject.

8. The method of claim 7, wherein the cardiomyopathy is hypertrophic cardiomyopathy, dilated cardiomyopathy, restrictive cardiomyopathy or arrhythmogenic right ventricular cardiomyopathy.

9. The method of claim 8, wherein the cardiomyopathy is hypertrophic cardiomyopathy.

10. The method of claim 7, wherein the expression level of NRIP is the expression level of protein, RNA or DNA of NRIP.

11. The method of claim 7, wherein the NRIP is a Z-disc protein.

12. The method of claim 7, wherein the NRIP interacts with ACTN2.

13. The method of claim 7, wherein the test sample and the control sample are blood, serum or plasma.

14. A method of screening an agent for treating cardiomyopathy, comprising providing a test agent to a test sample from a subject suffering from cardiomyopathy, and measuring an expression level of NRIP in test sample, wherein an increasing or equal amount in the expression level of NRIP in the test sample, relative to a control sample from a non-cardiomyopathy subject, is indicative of the test agent being the agent for treating cardiomyopathy.

15. The method of claim 14, wherein the cardiomyopathy is hypertrophic cardiomyopathy, dilated cardiomyopathy, restrictive cardiomyopathy or arrhythmogenic right ventricular cardiomyopathy.

16. The method of claim 15, wherein the cardiomyopathy is hypertrophic cardiomyopathy.

17. The method of claim 14, wherein the expression level of NRIP is the expression level of protein, RNA or DNA of NRIP.

18. The method of claim 14, wherein the NRIP is a Z-disc protein.

19. The method of claim 14, wherein the NRIP interacts with ACTN2.

20. The method of claim 14, wherein the test sample and the control sample are blood, serum, plasma or cardiac cell.
